(19) United States
(12) Patent Application Publication
(10) Pub. No.: US 2025/0275923 A1
(43) Pub. Date: Sep. 4, 2025

(54) SINGLE LAYER CHEWABLE TABLET COMPRISING CETIRIZINE

(71) Applicant: Kenvue Brands LLC, Summit, NJ (US)

(72) Inventor: Joel H. Waldman, Chalfont, PA (US)

(21) Appl. No.: 19/212,009

(22) Filed: May 19, 2025

Related U.S. Application Data
(63) Continuation of application No. 17/156,816, filed on Jan. 25, 2021, now abandoned.
(60) Provisional application No. 62/969,357, filed on Feb. 3, 2020.

(51) Int. Cl. A61K 9/20; A61K 9/00; A61K 31/495; A61K 45/06

(52) U.S. Cl. CPC A61K 9/2018; A61K 9/0056; A61K 9/2059; A61K 9/2072; A61K 9/2095; A61K 31/495; A61K 45/06

(57) ABSTRACT

The present invention relates to a single layer chewable tablet comprising cetirizine, an optical isomer, or a pharmaceutically active salt thereof and at least one polyol. The present invention also relates to a method of alleviating a sign or symptom of allergy by orally administering the same single layer chewable tablet.

### CROSS REFERENCE TO RELATED APPLICATIONS

The present application is a continuation of U.S. patent application Ser. No. 17/156,816 filed on Jan. 25, 2021, which claims priority to U.S. Provisional Application No. 62/969,357, filed Feb. 3, 2020, the disclosure of which is hereby incorporated by reference in its entirety.

### FIELD OF THE INVENTION

The present invention relates to a single layer chewable tablet comprising cetirizine, an optical isomer, or a pharmaceutically active salt thereof and at least one polyol. The present invention also relates to a method of alleviating a sign or symptom of allergy by orally administering the same single layer chewable tablet.

### BACKGROUND OF THE INVENTION

Cetirizine is a generic name for 2-[2-[4-[(4-chlorphenyl)phenylmethyl]-1-piperazinyl]ethoxy]-acetic acid and is typically provided as a dihydrochloride salt. Cetirizine is an orally active and selective H1-receptor antagonist currently prescribed for the treatment of seasonal allergies in patients aged 2 years and older. The current commercial products (Zyrtec™) include, but not limited to, a white, film-coated, immediate release oral tablet in 10 mg strength, a 10 mg uncoated orally disintegrating tablet (ODT) and a sweet flavored syrup containing cetirizine hydrochloride at a concentration of 1 mg/ml for pediatric use. European patents Nos. 058,146, 294,993, and 357,369 and also PCT Patent Application WO1992/002212 describe cetirizine formulations for the controlled or continuous release of cetirizine in the form of tablets and capsules. Oral formulations in the form of a cough syrup are disclosed in WO1994/008551.

For patients, such as children, who have difficulty swallowing conventional tablets or capsules, chewable tablets are widely used in the pharmaceutical industry. In addition, chewable tablets avoid mishaps that may occur with liquids, such as spillage and stains.

Polyols are used in tablets containing pharmaceutically active agents for various purposes. In chewable and fast dissolving tablets, polyols are frequently used as bulk filling, sweetening and taste masking agents. The pharmaceutically active agent cetirizine, however, is both bitter and highly susceptible to degradation by esterification with polyols. PCT Patent Application WO 03/059328 discloses that esterification of cetirizine can be controlled by creating a very dry environment and/or by physical separation of cetirizine and polyols in the tablet. U.S. Patent Application 2005/0038039 also discloses that when polyols of low molecular weight (molecular weight less than 950) are used with cetirizine in the molar ratio of polyol to cetirizine above 10, it leads to undesired reaction product. Hence, the polyol and the drug have been taken in separate layers of the bilayer tablet.

Surprisingly, Applicants have discovered that cetirizine, an optical isomer, or a pharmaceutically active salt thereof and at least one polyol can be formulated into a single layer chewable tablet. The resulting chewable tablet remains chemically and physically stable up to 6 months under accelerated conditions of 40° C. and 75% relative humidity (RH), up to 12 months under conditions of 30° C. and 65% relative humidity (RH) and 25° C. and 60% relative humidity (RH), up to 24 months under conditions of 30° C. and 65% relative humidity (RH) and 25° C. and 60% relative humidity (RH), and up to 36 months under conditions of 25° C. and 60% relative humidity (RH). This process further provides an economical method of making chewable tablets, suitable to various doses of cetirizine (e.g., 2.5 mg, 5 mg, or 10 mg), as it uses conventional equipment, commercially available excipients, and relatively simple process steps.

### SUMMARY OF THE INVENTION

The present invention provides a single layer chewable tablet comprising cetirizine, an optical isomer, or a pharmaceutically active salt thereof and at least one polyol. The present invention also provides a method for alleviating a sign or symptom of allergy by orally administering a single layer chewable tablet comprising cetirizine and or a pharmaceutically active salt thereof and at least one polyol.

In some embodiments, the at least one polyol is a sugar alcohol. Optionally, the sugar alcohol is selected from a group consisting of mannitol, xylitol, sorbitol, erythritol, lactitol, maltitol, isomalt or a mixture thereof. In some embodiments, the at least one polyol has a molecular weight of less than 1000. In some embodiments, the at least one polyol is mannitol.

In some embodiments, the single layer chewable tablet further comprises starch. Optionally, the at least one polyol and starch are preformed into a mixture. Optionally, the at least one polyol is about 70-90% by weight and the starch is about 10-30% by weight, relative to the total weight of the mixture.

In some embodiments, the single layer chewable tablet further comprises a cyclodextrin. Optionally, the cyclodextrin is selected from a group consisting of α-cyclodextrin, β-cyclodextrin, and γ-cyclodextrin. Optionally, the cyclodextrin is β-cyclodextrin. Optionally, the cyclodextrin is about 5%-30%, 10%-25%, or 12%-20% by weight, relative to the total weight of the chewable tablet. In some embodiments, the weight ratio of cetirizine and the cyclodextrin in the chewable tablet is about 1:1 to 1:10, 1:2 to 1:9, or 1:2.5 to 1:8.5.

In some embodiments, the chewable tablet remains stable up to 6 months under accelerated conditions of 40° C. and 75% relative humidity (RH). In some embodiments, the chewable tablet remains stable up to 12 months under conditions of 30° C. and 65% relative humidity (RH) and 25° C. and 60% relative humidity (RH). In some embodiments, the chewable tablet remains stable up to 24 months under conditions of 30° C. and 65% RH and 25° C. and 60% RH. In some embodiments, the chewable tablet remains stable up to 36 months under conditions of 25° C. and 60% RH.

In some embodiments, the chewable tablet has a hardness of about 2-12, 3-11, or 4-10 kp. In some embodiments, cetirizine is about 0.5%-20%, 0.5%-15%, 1%-10%, 1%-8%, 1%-6%, 1%-4%, or 1.5%-3.5% by weight, relative to the total weight of the chewable tablet.

In some embodiments, the chewable tablet further comprises additional pharmaceutically acceptable excipients. Optionally, the additional pharmaceutically acceptable excipients comprise fillers, adsorbents, binders, disintegrants, lubricants, glidants, sweeteners, superdisintegrants, flavor and aroma agents, antioxidants, texture enhancers, or mixtures thereof. Optionally, the fillers comprise monosaccharides, disaccharides, or mixtures thereof. Optionally, the disaccharides are lactose monohydrates. Optionally, the lactose monohydrates are about 5%-25% or 10-20% by weight, relative to the total weight of the chewable tablet.

In some embodiments, the chewable tablet further comprises a second active ingredient. Optionally, the second active ingredient is selected from the group consisting of phenylephrine, loratadine, fexofenadine, diphenhydramine, dextromethorphan, chlorpheniramine, chlophedianol, guaifenesin and pseudoephedrine.

In some embodiments, the chewable tablet is substantially free of coloring agents. Optionally, the coloring agents comprise azo dyes, quinopthalone dyes, triphenylmethane dyes, xanthene dyes, indigoid dyes, iron oxides, iron hydroxides, titanium dioxide, natural dyes, or mixtures thereof.

### DETAILED DESCRIPTION OF THE INVENTION

The present invention relates to a single layer chewable tablet comprising cetirizine, an optical isomer, or a pharmaceutically active salt thereof and at least one polyol. The present invention also relates to a method of alleviating a sign or symptom of allergy by orally administering the same single layer chewable tablet.

The following examples are provided to further illustrate the compositions and methods of the present invention. It should be understood that the present invention is not limited to the example described.

Unless defined otherwise, all technical and scientific terms used herein have the same meaning as commonly understood by one of ordinary skill in the art to which the invention belongs. Also, all publications, patent applications, patents, and other references mentioned herein are incorporated by reference. As used herein, all percentages are by weight unless otherwise specified.

In one aspect, the present disclosure provides a single layer chewable tablet (Composition 1) comprising cetirizine, an optical isomer, or a pharmaceutically active salt thereof and at least one polyol:
1.1 Composition 1, wherein the at least one polyol is a sugar alcohol.
1.2 Composition 1 or 1.1, wherein the at least one polyol has a molecular weight of less than 1000, less than 500, or less than 400.
1.3 Any of Compositions 1-1.2, wherein the sugar alcohol is selected from a group consisting of mannitol, xylitol, sorbitol, erythritol, lactitol, maltitol, isomalt or a mixture thereof.
1.4 Any of Compositions 1-1.3, wherein the at least one polyol is mannitol.
1.5 Any of Compositions 1-1.4, further comprising starch.
1.6 Any of Compositions 1-1.5, wherein the at least one polyol and the starch are preformed into a mixture.
1.7 Composition 1.6, wherein the at least one polyol and the starch are co-processed by wet granulation, dry granulation, spray drying or extrusion.
1.8 Composition 1.7, wherein the at least one polyol is about 70%-90% by weight and the starch is about 10%-30% by weight, relative to the total weight of the mixture.
1.9 Composition 1.8, wherein the at least one polyol is about 80% by weight and the starch is about 20% by weight, relative to the total weight of the mixture.
1.10 Any of Compositions 1-1.9, further comprising a cyclodextrin.
1.11 Any of Compositions 1-1.10, wherein the cyclodextrin is selected from a group consisting of α-cyclodextrin, β-cyclodextrin, and γ-cyclodextrin.
1.12 Composition 1.10 or 1.11, wherein the cyclodextrin is β-cyclodextrin.
1.13 Any of Compositions 1.10-1.12, wherein the cyclodextrin is about 5%-30%, 7%-27%, 10%-25%, 12%-20%, 13%-19%, or 14%-18% by weight, relative to the total weight of the chewable tablet.
1.14 Any of Compositions 1-1.13, wherein the chewable tablet remains stable up to 6 months under accelerated conditions of 40° C. and 75% relative humidity (RH) and/or up to 12 months under conditions of 30° C. and 65% relative humidity (RH) and 25° C. and 60% relative humidity (RH).
1.15 Any of Compositions 1-1.14, wherein the chewable tablet remains stable up to 24 months under conditions of 30° C. and 65% relative humidity (RH) and 25° C. and 60% relative humidity (RH).
1.16 Any of Compositions 1-1.15, wherein the chewable tablet remains stable up to 36 months under conditions of 25° C. and 60% relative humidity (RH).
1.17 Any of Compositions 1-1.16, wherein the chewable tablet has a hardness of about 2-12, 3-11, 4-10, 5-9, or 6-8 kp.
1.18 Any of Compositions 1-1.17, wherein cetirizine is about 0.5%-20%, 0.5%-15%, 1%-10%, 1%-8%, 1%-6%, 1%-5%, 1%-4%, or 1.5%-3.5% by weight, relative to the total weight of the chewable tablet.
1.19 Any of Compositions 1-1.18, wherein the weight ratio of cetirizine and the cyclodextrin in the chewable tablet is about 1:1 to 1:10, 1:2 to 1:9, or 1:2.5 to 1:8.5.
1.20 Any of Compositions 1-1.19, further comprising additional pharmaceutically acceptable excipients.
1.21 Composition 1.20, wherein the additional pharmaceutically acceptable excipients comprise fillers, adsorbents, binders, disintegrants, lubricants, glidants, sweeteners, superdisintegrants, flavor and aroma agents, antioxidants, texture enhancers, or mixtures thereof.
1.22 Composition 1.21, wherein the fillers comprise monosaccharides, disaccharides, or mixtures thereof.
1.23 Composition 1.22, wherein the disaccharides are lactose monohydrates.
1.24 Composition 1.23, wherein the lactose monohydrates are about 5%-25%, 6%-24%, 7%-23%, 8%-22%, 9%-21%, or 10%-20% by weight, relative to the total weight of the chewable tablet.
1.25 Any of Compositions 1-1.24, further comprising a second active ingredient.
1.26 Composition 1.25, wherein the second active ingredient is selected from the group consisting of phenylephrine, loratadine, fexofenadine, diphenhydramine, dextromethorphan, chlorpheniramine, chlophedianol, guaifenesin and pseudoephedrine.
1.27 Any of Compositions 1-1.26, wherein the chewable tablet is substantially free of coloring agents.
1.28 Composition 1.27, wherein the coloring agents comprise azo dyes, quinopthalone dyes, triphenylmethane dyes, xanthene dyes, indigoid dyes, iron oxides, iron hydroxides, titanium dioxide, natural dyes, or mixtures thereof.
1.29 Any of Compositions 1-1.28, wherein the chewable tablet is substantially free of superdisintegrants.
1.30 Composition 1.29, wherein the superdisintegrants are selected from a group consisting of crosslinked croscarmellose sodium (XL-CMC), crospovidone and sodium starch glycolate (SSG).
1.31 Any of Compositions 1-1.30, wherein the cetirizine in the chewable tablet is about 1-20 mg, 1-15 mg, 1-13 mg, 1-10 mg, 2-10 mg, 2.5-10 mg, 3-9.5 mg, 3.5-9 mg, 4-8.5 mg, 4.5-8 mg, 5-7.5 mg, 2.5 mg, 5 mg, or 10 mg.
1.32 Any of Compositions 1-1.31, further comprising sucralose.
1.33 Composition 1.32, wherein the sucralose is about 0.1%-5%, 0.1%-2%, 0.2%-2%, 0.2%-1.5%, 0.2%-1%, 0.3%-0.9%, 0.3%-0.8%, or 0.35%-0.7% by weight, relative to the total weight of the chewable tablet.

In another aspect, the present disclosure provides a method (Method 1) for alleviating a sign or symptom of allergy by orally administering a single layer chewable tablet comprising cetirizine and/or a pharmaceutically active salt thereof and at least one polyol:
1.1 Method 1, wherein the at least one polyol is a sugar alcohol.
1.2 Method 1 or 1.1, wherein the at least one polyol has a molecular weight of less than 1000, less than 500, or less than 400.
1.3 Any of Methods 1-1.2, wherein the sugar alcohol is selected from a group consisting of mannitol, xylitol, sorbitol, erythritol, lactitol, maltitol, isomalt or a mixture thereof.
1.4 Any of Methods 1-1.3, wherein the at least one polyol is mannitol.
1.5 Any of Methods 1-1.4, wherein the chewable tablet further comprises starch.
1.6 Any of Methods 1-1.5, wherein the at least one polyol and the starch are preformed into a mixture.
1.7 Method 1.6, wherein the at least one polyol and the starch are co-processed by wet granulation, dry granulation, spray drying or extrusion.
1.8 Method 1.7, wherein the at least one polyol is about 70%-90% by weight and the starch is about 10%-30% by weight, relative to the total weight of the mixture.
1.9 Method 1.8, wherein the at least one polyol is about 80% by weight and the starch is about 20% by weight, relative to the total weight of the mixture.
1.10 Any of Methods 1-1.9, wherein the chewable tablet comprises a cyclodextrin.
1.11 Any of Methods 1-1.10, wherein the cyclodextrin is selected from a group consisting of α-cyclodextrin, β-cyclodextrin, and γ-cyclodextrin.
1.12 Method 1.10 or 1.11, wherein the cyclodextrin is β-cyclodextrin.
1.13 Any of Methods 1.10-1.12, wherein the cyclodextrin is about 5%-30%, 7%-27%, 10%-25%, 12%-20%, 13%-19%, or 14%-18% by weight, relative to the total weight of the chewable tablet.
1.14 Any of Methods 1-1.13, wherein the chewable tablet remains stable up to 6 months under accelerated conditions of 40° C. and 75% relative humidity (RH) and/or up to 12 months under conditions of 30° C. and 65% relative humidity (RH) and 25° C. and 60% relative humidity (RH).
1.15 Any of Methods 1-1.14, wherein the chewable tablet remains stable up to 24 months under conditions of 30° C. and 65% relative humidity (RH) and 25° C. and 60% relative humidity (RH).
1.16 Any of Methods 1-1.15, wherein the chewable tablet remains stable up to 36 months under conditions of 25° C. and 60% relative humidity (RH).
1.17 Any of Methods 1-1.16, wherein the chewable tablet has a hardness of about 2-12, 3-11, 4-10, 5-9, or 6-8 kp.
1.18 Any of Methods 1-1.17, wherein cetirizine is about 0.5%-20%, 0.5%-15%, 1%-10%, 1%-8%, 1%-6%, 1%-5%, 1%-4%, or 1.5%-3.5% by weight, relative to the total weight of the chewable tablet.
1.19 Any of Methods 1-1.18, wherein the weight ratio of cetirizine and the cyclodextrin in the chewable tablet is about 1:1 to 1:10, 1:2 to 1:9, or 1:2.5 to 1:8.5.
1.20 Any of Methods 1-1.19, wherein the chewable tablet further comprises additional pharmaceutically acceptable excipients.
1.21 Method 1.20, wherein the additional pharmaceutically acceptable excipients comprise fillers, adsorbents, binders, disintegrants, lubricants, glidants, sweeteners, superdisintegrants, flavor and aroma agents, antioxidants, texture enhancers, or mixtures thereof.
1.22 Method 1.21, wherein the fillers comprise monosaccharides, disaccharides, or mixtures thereof.
1.23 Method 1.22, wherein the disaccharides are lactose monohydrates.
1.24 Method 1.23, wherein the lactose monohydrates are about 5%-25%, 6%-24%, 7%-23%, 8%-22%, 9%-21%, or 10%-20% by weight, relative to the total weight of the chewable tablet.
1.25 Any of Methods 1-1.24, wherein the chewable tablet comprises a second active ingredient.
1.26 Method 1.25, wherein the second active ingredient is selected from the group consisting of phenylephrine, loratadine, fexofenadine, diphenhydramine, dextromethorphan, chlorpheniramine, chlophedianol, guaifenesin and pseudoephedrine.
1.27 Any of Methods 1-1.26, wherein the chewable tablet is substantially free of coloring agents.
1.28 Method 1.27, wherein the coloring agents comprise azo dyes, quinopthalone dyes, triphenylmethane dyes, xanthene dyes, indigoid dyes, iron oxides, iron hydroxides, titanium dioxide, natural dyes, or mixtures thereof.
1.29 Any of Methods 1-1.28, wherein the chewable tablet is substantially free of superdisintegrants.
1.30 Method 1.29, wherein the superdisintegrants are selected from a group consisting of crosslinked croscarmellose sodium (XL-CMC), crospovidone and sodium starch glycolate (SSG).
1.31 Any of Methods 1-1.30, wherein the cetirizine in the chewable tablet is about 1-20 mg, 1-15 mg, 1-13 mg, 1-10 mg, 2-10 mg, 2.5-10 mg, 3-9.5 mg, 3.5-9 mg, 4-8.5 mg, 4.5-8 mg, 5-7.5 mg, 2.5 mg, 5 mg, or 10 mg.
1.32 Any of Methods 1-1.31, wherein the chewable tablet comprises sucralose.
1.33 Method 1.32, wherein the sucrose is about 0.1%-5%, 0.1%-2%, 0.2%-2%, 0.2%-1.5%, 0.2%-1%, 0.3%-0.9%, 0.3%-0.8%, or 0.35%-0.7% by weight, relative to the total weight of the chewable tablet.

In another aspect, the present disclosure provides use of a Composition of the present disclosure, e.g., a Composition described in any of the embodiments of Methods 1.1 to 1.33, in the manufacture of a medicament for use according to Method 1 or any of Methods 1.1-1.33.

In another aspect, the present disclosure provides a method (Method 2) for making a single layer chewable tablet comprising cetirizine, an optical isomer, or a pharmaceutically active salt thereof and at least one polyol: by providing cetirizine, an optical isomer, or a pharmaceutically active salt thereof and at least one polyol; dry blending cetirizine, an optical isomer, or a pharmaceutically active salt thereof, and the at least one polyol to form a mixture; and compressing the mixture:
2.1 Method 2, wherein the at least one polyol is a sugar alcohol.
2.2 Method 2 or 2.1, wherein the at least one polyol has a molecular weight of less than 1000, less than 500, or less than 400.
2.3 Any of Methods 2-2.2, wherein the sugar alcohol is selected from a group consisting of mannitol, xylitol, sorbitol, erythritol, lactitol, maltitol, isomalt or a mixture thereof.
2.4 Any of Methods 2-2.3, wherein the at least one polyol is mannitol.
2.5 Any of Methods 2-2.4, wherein the chewable tablet further comprises starch.
2.6 Any of Methods 2-2.5, wherein the at least one polyol and the starch are preformed into a mixture.
2.7 Method 2.6, wherein the at least one polyol and the starch are co-processed by wet granulation, dry granulation, spray drying or extrusion.
2.8 Method 2.7, wherein the at least one polyol is about 70%-90% by weight and the starch is about 10%-30% by weight, relative to the total weight of the mixture.
2.9 Method 2.8, wherein the at least one polyol is about 80% by weight and the starch is about 20% by weight, relative to the total weight of the mixture.
2.10 Any of Methods 2-2.9, wherein the chewable tablet comprises a cyclodextrin.
2.11 Any of Methods 2-2.10, wherein the cyclodextrin is selected from a group consisting of α-cyclodextrin, β-cyclodextrin, and γ-cyclodextrin.
2.12 Method 2.10 or 2.11, wherein the cyclodextrin is β-cyclodextrin.
2.13 Any of Methods 2.10-2.12, wherein the cyclodextrin is about 5%-30%, 7%-27%, 10%-25%, 12%-20%, 13%-19%, or 14%-18% by weight, relative to the total weight of the chewable tablet.
2.14 Any of Methods 2-2.13, wherein the chewable tablet remains stable up to 6 months under accelerated conditions of 40° C. and 75% relative humidity (RH) and/or up to 12 months under conditions of 30° C. and 65% relative humidity (RH) and 25° C. and 60% relative humidity (RH).
2.15 Any of Methods 2-2.14, wherein the chewable tablet remains stable up to 24 months under conditions of 30° C. and 65% relative humidity (RH) and 25° C. and 60% relative humidity (RH).
2.16 Any of Methods 2-2.15, wherein the chewable tablet remains stable up to 36 months under conditions of 25° C. and 60% relative humidity (RH).
2.17 Any of Methods 2-2.16, wherein the chewable tablet has a hardness of about 2-12, 3-11, 4-10, 5-9, or 6-8 kp.
2.18 Any of Methods 2-2.17, wherein cetirizine is about 0.5%-20%, 0.5%-15%, 1%-10%, 1%-8%, 1%-6%, 1%-5%, by weight, relative to the total weight of the chewable tablet.
2.19 Any of Methods 2-2.18, wherein the weight ratio of cetirizine and the cyclodextrin in the chewable tablet is about 1:1 to 1:10, 1:2 to 1:9, or 1:2.5 to 1:8.5.
2.20 Any of Methods 2-2.19, wherein the chewable tablet further comprises additional pharmaceutically acceptable excipients.
2.21 Method 2.20, wherein the additional pharmaceutically acceptable excipients comprise fillers, adsorbents, binders, disintegrants, lubricants, glidants, sweeteners, superdisintegrants, flavor and aroma agents, antioxidants, texture enhancers, or mixtures thereof.
2.22 Method 2.21, wherein the fillers comprise monosaccharides, disaccharides, or mixtures thereof.
2.23 Method 2.22, wherein the disaccharides are lactose monohydrates.
2.24 Method 2.23, wherein the lactose monohydrates are about 5%-25%, 6%-24%, 7%-23%, 8%-22%, 9%-21%, or 10%-20% by weight, relative to the total weight of the chewable tablet.
2.25 Any of Methods 2-2.24, wherein the chewable tablet comprises a second active ingredient.
2.26 Method 2.25, wherein the second active ingredient is selected from the group consisting of phenylephrine, loratadine, fexofenadine, diphenhydramine, dextromethorphan, chlorpheniramine, chlophedianol, guaifenesin and pseudoephedrine.
2.27 Any of Methods 2-2.26, wherein the chewable tablet is substantially free of coloring agents.
2.28 Method 2.27, wherein the coloring agents comprise azo dyes, quinopthalone dyes, triphenylmethane dyes, xanthene dyes, indigoid dyes, iron oxides, iron hydroxides, titanium dioxide, natural dyes, or mixtures thereof.
2.29 Any of Methods 2-2.28, wherein the chewable tablet is substantially free of superdisintegrants.
2.30 Method 2.29, wherein the superdisintegrants are selected from a group consisting of crosslinked croscarmellose sodium (XL-CMC), crospovidone and sodium starch glycolate (SSG).
2.31 Any of Methods 2-2.30, wherein the cetirizine in the chewable tablet is about 1-20 mg, 1-15 mg, 1-13 mg, 1-10 mg, 2-10 mg, 2.5-10 mg, 3-9.5 mg, 3.5-9 mg, 4-8.5 mg, 4.5-8 mg, 5-7.5 mg, 2.5 mg, 5 mg, or 10 mg.
2.32 Any of Methods 2-2.31, wherein the chewable tablet comprises sucralose.
2.33 Method 2.32, wherein the sucrose is about 0.1%-5%, 0.1%-2%, 0.2%-2%, 0.2%-1.5%, 0.2%-1%, 0.3%-0.9%, 0.3%-0.8%, or 0.35%-0.7% by weight, relative to the total weight of the chewable tablet.

Cetirizine is the compound [2-[4-[(4-chlorophenyl)phenylmethyl]-1-piperazinyl]ethoxy]acetic acid, including isomers thereof (such as 2-[2-[4-[(R)-(4-chlorophenyl)-phenyl-methyl]piperazin-1-yl]ethoxy]acetic acid known as levocetirizine), and pharmaceutically acceptable salts thereof (such as cetirizine dihydrochloride and levocetirizine dihydrochloride).

A polyol is a compound comprising two or more hydroxyl groups. Examples of polyols include, but are not limited to, sugars alcohols such as mannitol, xylitol, sorbitol, erythritol, lactitol, maltitol, and isomalt. In some embodiments, the polyol is about 40%-70%, 42%-68%, 44%-56%, 48%-54% by weight, relative to the total weight of the chewable tablet. In some embodiments, the polyol is mannitol. In some embodiments, the mannitol and starch are co-processed into a mixture, e.g., Pearlitol Flash. Pearlitol® Flash is commercially available from the Roquette Corporation.

Suitable cyclodextrins for use in the present invention include a, B, or γcyclodextrins, or an alkylated or hydroxyalkylated derivatives thereof, such as heptakis (2,6-di-o-methyl)-β-cyclodextrin (DIMEB), randomly methylated β-cyclodextrin (RAMEB), and hydroxypropyl β-cyclodextrin (HPBCD). A preferred cyclodextrin is β-cyclodextrin (available from Cerestar USA, Inc., Hammond, Ind. or from Roquette America, Inc., Keokuk. Iowa under the tradename Kleptose™).

Suitable excipients include, but are not limited to, fillers, adsorbents, binders, disintegrants, lubricants, glidants, sweeteners, superdisintegrants, flavor and aroma agents, antioxidants, texture enhancers, and mixtures thereof.

Suitable fillers include, but are not limited to, water-soluble compressible carbohydrates such as sugars (e.g., dextrose, sucrose, maltose, and lactose), starches (e.g., corn starch), sugar-alcohols (e.g., mannitol, sorbitol, maltitol, erythritol, and xylitol), starch hydrolysates (e.g., dextrins, and maltodextrins), and water insoluble plastically deforming materials (e.g., microcrystalline cellulose or other cellulosic derivatives), and mixtures thereof.

Mannitol is a desirable filler in tablets when taste is a factor as in chewable tablets. It is a white, odourless, crystalline powder, or free-flowing granules that is essentially inert and nonhygroscopic. It is commonly used as diluent in the manufacture of chewable tablet formulation because of its negative heat of solution, sweetness, and “mouth feel”. The popularity of mannitol as a suitable base in chewable tablet formulations is also attributed to its non-hygroscopic property. Mannitol also acts as a sweetening agent, and is said to be about 70% as sweet as sucrose.

Suitable adsorbents (e.g., to adsorb the liquid drug composition) include, but are not limited to, water-insoluble adsorbents such as dicalcium phosphate, tricalcium phosphate, silicified microcrystalline cellulose (e.g., such as distributed under the PROSOLV brand (Pen West Pharmaceuticals, Patterson, NY)), magnesium aluminometasilicate (e.g., such as distributed under the NEUSILIN™ brand (Fuji Chemical Industries (USA) Inc., Robbinsville, NJ), clays, silicas, bentonite, zeolites, magnesium silicates, hydrotalcite, veegum, and mixtures thereof.

Suitable binders include, but are not limited to, dry binders such as polyvinyl pyrrolidone and hydroxypropylmethylcellulose; wet binders such as water-soluble polymers, including hydrocolloids such as acacia, alginates, agar, guar gum, locust bean, carrageenan, carboxymethylcellulose, tara, gum arabic, tragacanth, pectin, xanthan, gellan, gelatin, maltodextrin, galactomannan, pusstulan, laminarin, scleroglucan, inulin, whelan, rhamsan, zooglan, methylan, chitin, cyclodextrin, chitosan, polyvinyl pyrrolidone, cellulosics, sucrose, and starches; and mixtures thereof.

Suitable disintegrants include, but are not limited to, sodium starch glycolate, cross-linked polyvinylpyrrolidone, cross-linked carboxymethylcellulose, starches, microcrystalline cellulose, and mixtures thereof.

Suitable lubricants include, but are not limited to, long chain fatty acids and their salts, such as magnesium stearate and stearic acid, talc, glycerides waxes, and mixtures thereof.

Suitable glidants include, but are not limited to, colloidal silicon dioxide.

Suitable coloring agents for use in a food or pharmaceutical product may be used in the present inventive composition. Typical coloring agents include, for example, azo dyes, quinopthalone dyes, triphenylmethane dyes, xanthene dyes, indigoid dyes, iron oxides, iron hydroxides, titanium dioxide, natural dyes, and mixtures thereof. More specifically, suitable colorants include, but are not limited to patent blue V, acid brilliant green BS, red 2G, azorubine, ponceau 4R, amaranth, D&C red 33, D&C red 22, D&C red 26, D&C red 28, D&C yellow 10, FD&C yellow 5, FD&C yellow 6, FD&C red 3, FD&C red 40, FD&C blue 1, FD&C blue 2, FD&C green 3, brilliant black BN, carbon black, iron oxide black, iron oxide red, iron oxide yellow, titanium dioxide, riboflavin, carotenes, antyhocyanines, turmeric, cochineal extract, clorophyllin, canthaxanthin, caramel, betanin, and mixtures thereof.

Suitable sweeteners include, but are not limited to, synthetic or natural sugars and high intensity sweeteners such as sucralose, saccharin, sodium saccharin, aspartame, acesulfame K or acesulfame, potassium acesulfame, thaumatin, glycyrrhizin, dihydrochalcone, alitame, miraculin, monellin, and stevside, and mixtures thereof. In one embodiment a high intensity sweetener is added to the pre-complexed granulation containing cetirizine and a polyol. In one embodiment a high intensity sweetener is added to the tablet matrix.

Suitable superdisintegrants include, but are not limited to, croscarmellose sodium, sodium starch glycolate and cross-linked povidone (crospovidone). In some embodiments, the tablet is substantially free of superdisintegrants.

As used herein, “substantially free of superdisintegrants” is defined as the weight % of the superdisintegrants in the chewable tablet, relative to the total weight of the chewable tablet, 10% or less, preferably 5% or less, preferably 2% or less, preferably 1% or less, preferably 0.5% or less, and more preferably 0.25% or less.

Suitable flavor and aroma agents include, but are not limited to, essential oils including distillations, solvent extractions, or cold expressions of chopped flowers, leaves, peel or pulped whole fruit containing mixtures of alcohols, esters, aldehydes and lactones; essences including either diluted solutions of essential oils, or mixtures of synthetic chemicals blended to match the natural flavor of the fruit (e.g., strawberry, raspberry, and black currant); artificial and natural flavors of brews and liquors (e.g., cognac, whisky, rum, gin, sherry, port, and wine); tobacco, coffee, tea, cocoa, and mint; fruit juices including expelled juice from washed, scrubbed fruits such as lemon, orange, and lime; mint; ginger; cinnamon; cacoc/cocoa; vanilla; liquorice; menthol; eucalyptus; aniseeds nuts (e.g., peanuts, coconuts, hazelnuts, chestnuts, walnuts, and colanuts); almonds; raisins; and powder, flour, or vegetable material parts including tobacco plant parts (e.g., the genus Nicotiana in amounts not contributing significantly to a level of therapeutic nicotine), and mixtures thereof.

Suitable antioxidants include, but are not limited to, tocopherols, ascorbic acid, sodium pyrosulfite, butylhydroxytoluene, butylated hydroxyanisole, edetic acid, and edetate salts, and mixtures thereof. Examples of preservatives include, but are not limited to, citric acid, tartaric acid, lactic acid, malic acid, acetic acid, benzoic acid, and sorbic acid, and mixtures thereof.

Suitable secondary active ingredients include other pharmaceuticals, minerals, vitamins, other nutraceuticals, and mixtures thereof. Suitable pharmaceuticals include analgesics, anti-inflammatory agents, antiarthritics, anesthetics, antihistamines, antitussives, antibiotics, anti-infective agents, antivirals, anticoagulants, antidepressants, antidiabetic agents, antiemetics, antiflatulents, antifungals, antispasmodics, appetite suppressants, bronchodilators, cardiovascular agents, central nervous system agents, central nervous system stimulants, decongestants, diuretics, expectorants, gastrointestinal agents, migraine preparations, motion sickness products, mucolytics, muscle relaxants, osteoporosis preparations, polydimethylsiloxanes, respiratory agents, sleep aids, urinary tract agents and mixtures thereof.

In one embodiment, the secondary active agent may be selected from bisacodyl, famotidine, ranitidine, cimetidine, prucalopride, diphenoxylate, loperamide, lactase, mesalamine, bismuth, antacids, and pharmaceutically acceptable salts, esters, isomers, and mixtures thereof.

In another embodiment, the secondary active agent may be selected from acetaminophen, acetyl salicylic acid, diclofenac, cyclobenzaprine, meloxicam, cox-2 inhibitors such as rofecoxib and celecoxib, codeine, oxycodone, hydrocodone, tramadol, and pharmaceutically acceptable salts, esters, isomers, and mixtures thereof.

In another embodiment, the active agent may be selected from pseudoephedrine, phenylepherine, methocarbamol, doxylamine, guaifenesin, antacids, simethicone, cyclobenzaprine, chloroxazone, glucosamine, chondroitin, phenylpropanolamine, chlorpheniramine, dextromethorphan, diphenhydramine, astemizole, terfenadine, fexofenadine, loratadine, cetirizine, mixtures thereof and pharmaceutically acceptable salts, esters, isomers, and mixtures thereof.

The secondary active ingredient(s) are present in the dosage form in a therapeutically effective amount, which is an amount that produces the desired therapeutic response upon oral administration and can be readily determined by one skilled in the art. In determining such amounts, it is well known in the art that various factors must be considered that include, but are not limited to the particular active ingredient being administered, the bioavailability characteristics of the active ingredient, the dose regime, and the age and weight of the patient.

Suitable dosage forms may be pharmaceutical drug delivery systems, including those for oral administration, buccal administration, and the like. In one embodiment, the dosage forms of the present invention are considered to be solid; however, they may contain liquid or semi-solid components. In another embodiment, the dosage form is an orally administered system for delivering a pharmaceutical active ingredient to the gastro-intestinal tract of a human. In yet another embodiment, the dosage form is an orally administered “placebo” system containing pharmaceutically inactive ingredients, and the dosage form is designed to have the same appearance as a particular pharmaceutically active dosage form, such as may be used for control purposes in clinical studies to test, for example, the safety and efficacy of a particular pharmaceutically active ingredient. In one embodiment, the dosage form contains all active ingredients within the same solid, semi-solid, or liquid forms. In another embodiment, the dosage form contains the active ingredients in one or more solid, semi-solid, or liquid forms. In one embodiment, the dosage form is an orally disintegrating tablet. In one embodiment, the dosage form is a chewable tablet that is beneficial to those who have difficulty in swallowing a tablet. In one embodiment, the dosage form has the advantage of preventing discolor on stability.

In some embodiments, the chewable tablet remains stable at 1, 3, and 6 months under accelerated conditions of 40° C. and 75% relative humidity (RH). In some embodiments, the chewable tablet remains stable at 1, 2, 3, 6, 12, 18 and 24 months under conditions of 30° C. and 65% relative humidity (RH). In some embodiments, the chewable tablet remains stable at 1, 3, 6, 12, 18, 24, 30 and 36 months under conditions of 25° C. and 60% relative humidity (RH).

In some embodiments, the chewable tablet remains stable at 1, 3, and 6 months under accelerated conditions of 40° C. and 75% relative humidity (RH): e.g., at least 75% of the active ingredient released, at least 80% of the active ingredient released, at least 85% of the active ingredient released, at least 90% of the active ingredient released, at least 95% of the active ingredient released, at least 96% of the active ingredient released, at least 97% of the active ingredient released, at least 98% of the active ingredient released, or at least 99% of the active ingredient released within 30 minutes @ 50 RPM dissolution.

In some embodiments, the chewable tablet remains stable at 1, 2, 3, 6, 12, 18 and 24 months under conditions of 30° C. and 65% relative humidity (RH): e.g., at least 75% of the active ingredient released, at least 80% of the active ingredient released, at least 85% of the active ingredient released, at least 90% of the active ingredient released, at least 95% of the active ingredient released, at least 96% of the active ingredient released, at least 97% of the active ingredient released, at least 98% of the active ingredient released, or at least 99% of the active ingredient released within 30 minutes @ 50 RPM dissolution.

In some embodiments, the chewable tablet remains stable at 1, 3, 6, 12, 18, 24, 30 and 36 months under conditions of 25° C. and 60% relative humidity (RH): e.g., at least 75% of the active ingredient released, at least 80% of the active ingredient released, at least 85% of the active ingredient released, at least 90% of the active ingredient released, at least 95% of the active ingredient released, at least 96% of the active ingredient released, at least 97% of the active ingredient released, at least 98% of the active ingredient released, or at least 99% of the active ingredient released within 30 minutes @ 50 RPM dissolution.

In some embodiments, the chewable tablet remains stable at 1, 3, and 6 months under accelerated conditions of 40° C. and 75% relative humidity (RH): e.g., at least 75% of the active ingredient released, at least 80% of the active ingredient released, at least 85% of the active ingredient released, at least 90% of the active ingredient released, at least 95% of the active ingredient released, at least 96% of the active ingredient released, at least 97% of the active ingredient released, at least 98% of the active ingredient released, or at least 99% of the active ingredient released within 20 minutes @ 50 RPM dissolution.

In some embodiments, the chewable tablet remains stable at 1, 2, 3, 6, 12, 18 and 24 months under conditions of 30° C. and 65% relative humidity (RH): e.g., at least 75% of the active ingredient released, at least 80% of the active ingredient released, at least 85% of the active ingredient released, at least 90% of the active ingredient released, at least 95% of the active ingredient released, at least 96% of the active ingredient released, at least 97% of the active ingredient released, at least 98% of the active ingredient released, or at least 99% of the active ingredient released within 20 minutes @ 50 RPM dissolution.

In some embodiments, the chewable tablet remains stable at 1, 3, 6, 12, 18, 24, 30 and 36 months under conditions of 25° C. and 60% relative humidity (RH): e.g., at least 75% of the active ingredient released, at least 80% of the active ingredient released, at least 85% of the active ingredient released, at least 90% of the active ingredient released, at least 95% of the active ingredient released, at least 96% of the active ingredient released, at least 97% of the active ingredient released, at least 98% of the active ingredient released, or at least 99% of the active ingredient released within 20 minutes @ 50 RPM dissolution.

### Method of Making Tablets

The manufacture of chewable tablets basically follows the design/pattern for conventional tablets. The wet granulation method is frequently employed. However, other methods such as direct compression, and dry granulation or slugging/pre-compression may be employed depending on the nature of the excipients being used.

Wet granulation method is a process of size enlargement in which fine powder particles are agglomerated or brought together into larger, strong and relatively permanent structure called granules using a suitable non-toxic granulating fluid such as water, isopropanol or ethanol (or mixtures thereof). The granulating fluid can be used alone or as a solvent containing the active ingredient, binder or granulating agent. The choice of the granulating fluid depends greatly on the properties of the materials to be granulated. Powder mixing, in conjunction with the cohesive properties of the granulating agent, enables the formation of granules. The characteristics and performance of the final product greatly depends on the extent to which the powder particles interact with each other to form aggregates (granules).

Direct compression (or direct compaction) is used to define the process by which tablets are compressed directly from powdered active drug substance and suitable excipients into a firm compact without employing the process of granulation.

### Uses of Tablets

An allergy occurs when the body's immune system sees a substance as harmful and overreacts to it. The symptoms that result are an allergic reaction. Some of the symptoms of an allergic reaction include, but not limited to, itchy, watery eyes; itchy nose; sneezing; running nose; rashes; hives; stomach cramps; vomiting; diarrhea; bloating, swelling; redness; pain; tongue swelling; cough; throat closing; and wheezing. The substances that cause allergic reactions are allergens. Allergens can get into your body many ways to cause an allergic reaction. There are many safe prescription and over-the-counter medicines to relieve allergy symptoms, including but not limited to, nasal corticosteroids, antihistamines, mast cell stabilizers, decongestants, corticosteroid creams or ointments, oral corticosteroids and epinephrine.

### EXAMPLES

### Example 1: Single Layer Chewable Tablet Formulations

### Formulations A, B, and C:

Part A: Blending: The blend(s) were prepared as follows:
1. Batches up to 800 kg were prepared according to the blend formula in Table 1.
2. The cetirizine, lactose monohydrate & a portion of the Pearlitol Flash were preblended until sufficiently uniform. Pearlitol Flash is commercially available from the Roquette Corporation.
3. The other materials, except the lubricant and a small portion of the Pearlitol Flash, were added and blended for an additional period.
4. The lubricant and remaining Pearlitol Flash were added and blended for additional 5 minutes.

Part B: Compression: The blends in Table 1 were compressed into tablets using round tooling. Formula A contains 2.5 mg cetirizine; Formula B contains 5 mg cetirizine; and Formula C contains 10 mg cetirizine.

TABLE 1
Single Layer Chewable Tablet Formulations
with Mannitol/Starch Blend

| Ingredients | Wt Percent |
|---|---|
| Cetirizine HCl | e.g., 0.5%-5% |
| Beta-Cyclodextrin | e.g., 5%-30% |
| Mannitol USP & Starch NF | e.g., 30%-60% |
| (Pearlitol Flash) | |
| Lactose Monohydrate NF | e.g., 5%-25%, |
| Silicified Microcrystalline | e.g., 2%-10% |
| Cellulose | |
| Sucralose NF Powder | e.g., 0.1%-1%, |
| Flavor | e.g., 0.1%-1% |
| Magnesium Stearate NF | e.g., 0.1%-1% |
| Total (% w/w) | 100% |

### Example 2: Single Layer Chewable Tablet Formulations without Mannitol & Starch Blend

Part A: The formulations D containing 5 mg cetirizine and E containing 10 mg cetirizine were produced for comparison purposes.

Part B: Tablets were prepared using the blends from Part A. 5 mg cetirizine tablets were compressed to a hardness of approximately 7.0 kp and thickness of approximately 4.8 mm. 10 mg cetirizine tablets were compressed to a hardness of approximately 7.5 kp and a thickness of approximately 5.6 mm.

TABLE 2
Single Layer Chewable Tablet Formulations
without Mannitol & Starch Blend

| Ingredients | Wt Percent |
|---|---|
| Cetirizine HCl | e.g., 0.5%-5% |
| Beta-Cyclodextrin | e.g., 5%-30% |
| (Kleptose) | |
| Acesulfame Potassium NF | e.g., 0.5%-2% |
| Colloidal Silicon Dioxide | e.g., 0.1%-1% |
| NF | |
| Microcrystalline Cellulose | e.g., 5%-15% |
| NF (Avicel PH102) | |
| Lactose Monohydrate NF | e.g., 5%-20% |
| (Fast-Flo 316) | |
| Flavor 1 | e.g., 0.1%-1% |
| Flavor 2 | e.g., 0.10%-1% |
| Colorant 1 | e.g., 0.10%-1% |
| Colorant 2 | e.g., 0.05%-1% |
| Magnesium Stearate NF | e.g., 0.5%-5% |
| 2257 | |
| Mannitol USP | e.g., 40%-60% |
| Total (% w/w) | 100% |

### Example 3: Dissolution on Stability

The following formulations were evaluated for dissolution and dissolution on stability under accelerated conditions of 40° C. and 75% relative humidity (RH).

The samples were tested in 900 mL of water using USP Apparatus 2 (paddles) at 50 RPM. Samples were pulled at respective timepoints and analyzed using an HPLC equipped with a UV detector set at 230 nm. A 4.6 mm×25 cm column was utilized with a flow rate of 1 ml/min and injection volume of 50 μL, and a mobile phase of 50:50 Acetonitrile and water and an adjusted pH of 3.5. Table 3 shows that Formulas A-C at the presence of the mannitol and starch blend remain stable up to 6 months under accelerated conditions of 40° C. and 75% relative humidity (RH). Formulas A-C are more stable than Formulas D-E without mannitol and starch blend, but mannitol only. Tables 4 and 5 also show that Formulas A-C at the presence of the mannitol and starch blend remain stable up to 12 months under conditions of 25° C./60% RH and 30° C./65% RH with 30 minutes @50 RPM dissolution, respectively. Table 6 shows that Formulas A-C at the presence of the mannitol and starch blend remain stable up to 6 months under accelerated conditions of 40° C. and 75% relative humidity (RH) with 20 minutes @ 50 RPM dissolution. Tables 7 and 8 show that Formulas A-C at the presence of the mannitol and starch blend remain stable up to 12 months under conditions of 25° C./60% RH and 30° C./65% RH with 20 minutes @ 50 RPM dissolution, respectively.

TABLE 3
Dissolution Stability under 40° C./75% RH with
30 minutes @ 50 RPM dissolution.

| | | Initial | 1 month | 3 months | 6 months |
|---|---|---|---|---|---|
| Formula A | Mean | 97 | 98 | 99 | 98 |
| | Min | 94 | 93 | 93 | 91 |
| | Max | 101 | 103 | 103 | 103 |
| Formula B | Mean | 98 | 98 | 97 | 99 |
| | Min | 96 | 96 | 93 | 97 |
| | Max | 99 | 100 | 100 | 102 |
| Formula C | Mean | 97 | 97 | 96 | 97 |
| | Min | 94 | 93 | 95 | 95 |
| | Max | 99 | 100 | 98 | 98 |
| Formula D | Mean | 97 | 80 | 78 | 73 |
| | Min | 93 | 71 | 69 | 63 |
| | Max | 105 | 88 | 93 | 84 |
| Formula E | Mean | 98 | 97 | 94 | 88 |
| | Min | 96 | 93 | 88 | 78 |
| | Max | 99 | 99 | 100 | 94 |

Results are an average 6 vessels (n = 6)

TABLE 4
Dissolution Stability under 25° C./60% RH with
30 minutes @ 50 RPM dissolution.

| | | Initial | 3 months | 6 months | 9 months | 12 months |
|---|---|---|---|---|---|---|
| Formula A | Mean | 97 | 99 | 98 | 99 | 96 |
| | Min | 94 | 95 | 93 | 93 | 92 |
| | Max | 101 | 105 | 102 | 103 | 98 |
| Formula B | Mean | 98 | 99 | 97 | 98 | 100 |
| | Min | 96 | 96 | 94 | 92 | 98 |
| | Max | 99 | 101 | 98 | 104 | 103 |
| Formula C | Mean | 97 | 97 | 98 | 97 | 98 |
| | Min | 94 | 96 | 96 | 95 | 96 |
| | Max | 99 | 99 | 101 | 100 | 100 |

TABLE 5
Dissolution Stability under 30° C./65% RH with
30 minutes @ 50 RPM dissolution.

| | | Initial | 3 months | 6 months | 9 months | 12 months |
|---|---|---|---|---|---|---|
| Formula A | Mean | 97 | 103 | 98 | 98 | 101 |
| | Min | 94 | 95 | 95 | 95 | 96 |
| | Max | 101 | 108 | 105 | 105 | 104 |
| Formula B | Mean | 98 | 99 | 99 | 99 | 98 |
| | Min | 96 | 94 | 96 | 95 | 95 |
| | Max | 99 | 111 | 103 | 103 | 102 |
| Formula C | Mean | 97 | 97 | 98 | 97 | 97 |
| | Min | 94 | 94 | 95 | 94 | 94 |
| | Max | 99 | 100 | 100 | 100 | 99 |

TABLE 6
Dissolution Stability under 40° C./75% RH with
20 minutes @ 50 RPM dissolution.

| | | Initial | 1 month | 3 months | 6 months |
|---|---|---|---|---|---|
| Formula A | Average | 97 | 98 | 98 | 98 |
| | Min | 95 | 93 | 93 | 91 |
| | Max | 98 | 103 | 103 | 102 |
| Formula B | Average | 98 | 98 | 97 | 98 |
| | Min | 96 | 96 | 93 | 96 |
| | Max | 100 | 99 | 99 | 101 |
| Formula C | Average | 96 | 96 | 95 | 95 |
| | Min | 94 | 92 | 94 | 93 |
| | Max | 98 | 99 | 96 | 96 |

TABLE 7
Dissolution Stability under 25° C./60% RH with
20 minutes @ 50 RPM dissolution.

| | | Initial | 3 months | 6 months | 9 months | 12 months |
|---|---|---|---|---|---|---|
| Formula | Average | 97 | 100 | 97 | 100 | 98 |
| A | Min | 95 | 96 | 93 | 94 | 90 |
| | Max | 98 | 105 | 101 | 104 | 103 |
| Formula | Average | 98 | 99 | 97 | 98 | 100 |
| B | Min | 96 | 97 | 93 | 92 | 98 |
| | Max | 100 | 100 | 98 | 104 | 103 |
| Formula | Average | 96 | 97 | 96 | 96 | 97 |
| C | Min | 94 | 96 | 95 | 95 | 93 |
| | Max | 98 | 98 | 99 | 99 | 99 |

TABLE 8
Dissolution Stability under 30° C./65% RH with
20 minutes @ 50 RPM dissolution.

| | | Initial | 3 months | 6 months | 9 months | 12 months |
|---|---|---|---|---|---|---|
| Formula | Average | 97 | 102 | 98 | 98 | 102 |
| A | Min | 95 | 96 | 92 | 95 | 97 |
| | Max | 98 | 107 | 103 | 102 | 105 |
| Formula | Average | 98 | 98 | 98 | 98 | 97 |
| B | Min | 96 | 94 | 96 | 94 | 93 |
| | Max | 100 | 111 | 102 | 102 | 102 |
| Formula | Average | 96 | 97 | 96 | 97 | 97 |
| C | Min | 94 | 95 | 86 | 94 | 95 |
| | Max | 98 | 101 | 99 | 99 | 98 |

### Example 4: Cetirizine (Active Ingredient) Stability

The stability of the active ingredient was evaluated for the samples from Formulas A-E. Samples were analyzed using an HPLC (or UPLC) equipped with a UV detector set at 230 nm. A 4.6 mm×25 cm column was utilized with a flow rate of 1 mL/min and injection volume of 50 μL, and a mobile phase of 50:50 Acetonitrile and water and an adjusted pH of 3.5 against a cetirizine reference standard at 100%. Formulas A-C have degraded more slowly than Formulas D-E under accelerated stability conditions of 40° C. and 75% RH.

TABLE 9
Active Ingredient Stability: Total Degradation Products under
Accelerated Stability Conditions of 40° C. and 75% RH

| | Initial | 1 month | 3 months | 6 months |
|---|---|---|---|---|
| Formula A | 0.01 | 0.15 | 0.52 | 0.69 |
| | | | (0.45 excluding | (0.56 excluding |
| | | | sucralose ester) | sucralose ester) |
| Formula B | 0.01 | 0.16 | 0.46 | 0.65 |
| | | | (0.38 excluding | (0.53 excluding |
| | | | sucralose ester) | sucralose ester) |
| Formula C | 0.01 | 0.173 | 0.50 | 0.73 |
| | | | (0.40 excluding | (0.58 excluding |
| | | | sucralose ester) | sucralose ester) |
| Formula D | 0.06 | 0.24 | 0.60 | 1.29 |
| Formula E | 0.12 | 0.24 | 0.45 | 1.05 |

### Example 5: Pharmacokinetic Data and Bioequivalence Data Under Different Treatments

This was a randomized, single-dose, 4-treatment crossover bioequivalence and food effect study. The study was conducted in 2 parts. Part 1 of the study had a randomized, 4-way crossover study design in which 40 healthy subjects, aged 18 to 55 years, were randomized to 4 sequences of Treatments A, B, D and E over consecutive periods. No less than approximately 40% of either gender was to be represented in the study population. Part 2 of the study assessed a potential food effect in which all subjects were administered Treatment C in the fifth period.

Different regimens used in the studies include the following:
1. Regimen for Treatment A: Single dose of 10 mg cetirizine as a chewable tablet (e.g., Formula C), administered orally after a 10 hour overnight fast and followed with 240 mL ambient water. Subjects were instructed to chew the tablet completely before swallowing.
2. Regimen for Treatment B: Single dose of 10 mg cetirizine as a chewable tablet (e.g., Formula C), administered orally after a 10 hour overnight fast without water. Subjects were instructed to chew the tablet completely before swallowing.
3. Regimen for Treatment C: Single dose of 10 mg cetirizine as a chewable tablet (e.g., Formula C), administered orally after a 10 hour overnight fast and 30 minutes after the start of the standard high-fat breakfast and followed with 240 mL ambient water. Subjects were instructed to chew the tablet completely before swallowing.
4. Regimen for Treatment D: Single dose of currently marketed US 10 mg cetirizine as IR tablet (Zyrtec®), administered orally after a 10-hour overnight fast and followed with 240 mL ambient water.
5. Regimen for Treatment E: Single dose of currently marketed EU/Australian 10 mg cetirizine film coated tablet (Reactine®) administered orally after a 10 hour overnight fast and followed with 240 mL ambient water.
   Duration of treatment: A single 10 mg oral dose of cetirizine was administered under fasting conditions (Treatment A, B, D and E) or fed conditions (Treatment C) in each study period. The drug administrations were separated by a wash-out of 7 calendar days. In each study period, 16 blood samples for PK measurements were taken before dosing and at 0.25, 0.5, 0.75, 1, 1.25, 1.5, 2, 3, 4, 6, 8, 12, 24, 28 and 32 hours after drug administration. Plasma was harvested and quantified for cetirizine using a validated analytical method.

Part 1: Statistical inference of cetirizine was based on a bioequivalence approach. For example, the ratios of geometric Least-Squares means (LSmeans) with corresponding 90% confidence intervals, calculated from the exponential of the difference between the Test and Reference products for the In-transformed parameters Cmax, AUC_{0-T} and AUC_{0-∞}, were all to be within the 80.00 to 125.00% bioequivalence range comparing Treatment A vs D, A vs E, B vs D, B vs E and D vs E, respectively.

Part 2: Statistical inference of food effects on cetirizine bioavailability used the following standards: The ratios of geometric LSmeans with corresponding 90% confidence intervals, calculated from the exponential of the difference between the Test and Reference products for the In-transformed parameters Cmax, AUC_{0-T} and AUC_{0-∞}, were all to be within the 80.00 to 125.00% range comparing Treatment C vs A.

Mathematical Model and Statistical Methods of Pharmacokinetic Parameters: The main absorption and disposition parameters were calculated using a non-compartmental approach with a log-linear terminal phase assumption. The trapezoidal rule was used to estimate area under the curve. The terminal phase estimation was based on maximizing the coefficient of determination. The statistical analyses of Cmax, AUC_{0-T} and AUC_{0-∞} were based on parametric ANOVA models of the In-transformed pharmacokinetic parameters; the two-sided 90% confidence intervals of the ratios of geometric means were, in each case, based on the fitted model and calculated through exponentiation.

A single center, randomized, single dose, laboratory-blinded, four-way, crossover comparative bioavailability and food effect study in 40 healthy male and female subjects. The rate and extent of absorption of cetirizine were assessed and compared following a single dose (1×10 mg) of the Test and the Reference formulations. The bioavailability of cetirizine was equivalent across all Test and Reference comparisons under fasting conditions. There was a food effect observed for cetirizine Cₘₐₓ. However, no significant food effect was observed for AUC_{0-T} or AUC_{0-∞}. The results from evaluable data from 40 subjects are presented in Table 7.

TABLE 7
Pharmacokinetic Parameters

| | Treatment A | | Treatment B | | Treatment C | |
|---|---|---|---|---|---|---|
| | (n = 39) | | (n = 40) | | (n = 38) | |
| PARAMETER | MEAN | C.V. (%) | MEAN | C.V. (%) | MEAN | C.V. (%) |
| Cₘₐₓ (ng/mL) | 330.26 | (20.9) | 326.17 | (21.1) | 183.69 | (16.6) |
| Tₘₐₓ (hours)^{a} | 0.75 | (0.50-1.50) | 1.25 | (0.50-3.00) | 4.00 | (1.03-6.00) |
| AUC_{0-T} (ng h/mL) | 2561.99 | (16.6) | 2592.22 | (17.4) | 2312.50 | (19.6) |
| AUC_{0-∞} (ng h/mL) | 2689.14 | (17.7) | 2716.42 | (18.1) | 2454.32 | (21.2) |
| AUC_{% Extrap}(%) | 4.49 | (60.9) | 4.38 | (62.0) | 5.41 | (56.0) |
| λ_{z} (hours⁻¹) | 0.1028 | (19.5) | 0.1057 | (19.2) | 0.1029 | (19.9) |
| T_{half} (hours) | 7.01 | (20.9) | 6.82 | (21.2) | 7.00 | (20.6) |

| | |
|---|---|
| Treatment D | Treatment E |
| (n = 40) | (n = 40) |

| | MEAN | C.V. (%) | MEAN | C.V. (%) |
|---|---|---|---|---|
| Cₘₐₓ (ng/mL) | 325.54 | (22.0) | 331.76 | (19.0) |
| Tₘₐₓ (hours)^{a} | 1.00 | (0.50-2.02) | 0.75 | (0.50-1.55) |
| AUC_{0-T} (ng h/mL) | 2546.89 | (18.1) | 2559.48 | (17.9) |
| AUC_{0-∞} (ng h/mL) | 2671.01 | (19.8) | 2679.15 | (19.1) |
| AUC_{% Extrap}(%) | 4.31 | (70.1) | 4.22 | (63.2) |
| λ_{z} (hours⁻¹) | 0.1070 | (20.3) | 0.1070 | (21.0) |
| T_{half} (hours) | 6.78 | (23.6) | 6.77 | (22.4) |

^{a}Median and range are presented

The results presented show that the criteria used to assess bioequivalence between the Test and Reference formulations under fasted conditions were all fulfilled. The Test to Reference estimated ratios of geometric LSmeans and corresponding 90% confidence intervals for Cmax, AUC_{0-T} and AUC_{0-∞} were all within the bioequivalence acceptance range of 80.00 to 125.00%.

Cetirizine Chewable Tablet, Fasted With Water (Treatment A) vs Zyrtec® IR tablet US Reference Fasted with Water (Treatment D): The Cₘₐₓ and AUC distributions were similar between Treatment A and Treatment D, with estimated geometric mean ratios and 90% CIs of 102.12% (98.02-106.40%), 100.67% (98.23%-103.17%) and 100.79% (98.26-103.38%) for Cₘₐₓ, AUC_{0-T} and AUC_{0-∞}, respectively. Hence, cetirizine chewable tablet with water (Treatment A) was judged to be bioequivalent to Zyrtec® tablet US reference (Treatment D), under fasting conditions.

Cetirizine Chewable Tablet, Fasted With Water (Treatment A) vs Reactine® IR tablet Australian Reference Fasted with Water (Treatment E): The Cmax and AUC distributions were similar between Treatment A and Treatment E, with estimated geometric mean ratios and 90% CIs of 99.72% (95.55-104.07%) and 100.06% (97.19%-103.01%) for Cₘₐₓ and AUC_{0-T} respectively. Hence, cetirizine chewable tablet with water (Treatment A) was judged to be bioequivalent to Reactine® tablet EU and Australian reference (Treatment E), under fasting conditions.

Cetirizine Chewable Tablet, Fasted Without Water (Treatment B) vs Zyrtec® IR tablet US Reference Fasted with Water (Treatment D): The Cₘₐₓ and AUC distributions were similar between Treatment B and Treatment D, with estimated geometric mean ratios and 90% CIs of 100.59% (96.58-104.77%), 101.96% (99.51%-104.47%) and 102.02% (99.48-104.63%) for Cₘₐₓ, AUC_{0-T} and AUC_{0-∞}, respectively. Hence, cetirizine chewable tablet without water (Treatment B) was bioequivalent to Zyrtec® tablet US reference (Treatment D), under fasting conditions.

Cetirizine Chewable Tablet, Fasted Without Water (Treatment B) vs Reactine® IR tablet Australian Reference Fasted with Water (Treatment E): The Cₘₐₓ and AUC distributions were similar between Treatment B and Treatment E, with estimated geometric mean ratios and 90% CIs of 98.10% (94.31%-102.05%) and 101.33% (98.82%-103.90%) for Cₘₐₓ and AUC_{0-T}, respectively. Hence, cetirizine chewable tablet without water (Treatment B) was judged to be bioequivalent to Reactine® tablet EU and Australian reference (Treatment E), under fasting conditions.

Zyrtec® IR tablet US Reference Fasted with Water (Treatment D) vs Reactine® IR tablet Australian Reference Fasted with Water (Treatment E): The Cₘₐₓ and AUC distributions were similar between Treatment D and Treatment E with estimated geometric mean ratios and 90% CIs of 97.52% (93.25%-101.99%) and 99.38% (96.94%-101.88%) for Cₘₐₓ and AUC_{0-T} respectively. Hence, Zyrtec® IR tablet US Reference (Treatment D) was judged to be bioequivalent to Reactine® tablet Australian reference (Treatment E), under fasting conditions.

Cetirizine Chewable Tablet, Fed With Water (Treatment C) vs Cetirizine Chewable Tablet, Fasted With Water (Treatment A): The Cₘₐₓ values were on average lower following Treatment C compared to Treatment A, with an estimated geometric mean ratio and 90% CI of 55.95% (53.63-58.38%). This difference was substantially less pronounced for the AUC values with estimated geometric mean ratios and 90% CIs of 90.25% (87.79%-92.78%) and 91.27% (88.60%-94.02%) for AUC_{0-T} and AUC_{0-∞}, respectively. Therefore, a food effect was observed for cetirizine Cₘₐₓ while for AUC_{0-T} and AUC_{0-∞}, the bioequivalence criterion was fulfilled, when cetirizine chewable tablet was administered following a high-fat breakfast with water.

1. A method for manufacturing a single layer chewable tablet comprising cetirizine, an optical isomer, or a pharmaceutically active salt thereof and at least one polyol.

2. The method for manufacturing a single layer chewable tablet of claim 1, wherein the at least one polyol is a sugar alcohol.

3. The method for manufacturing a single layer chewable tablet of claim 1, wherein the at least one polyol has a molecular weight of less than 1000.

4. The method for manufacturing a single layer chewable tablet of claim 1, wherein the sugar alcohol is selected from a group consisting of mannitol, xylitol, sorbitol, erythritol, lactitol, maltitol, isomalt or a mixture thereof.

5. The method for manufacturing a single layer chewable tablet of claim 1, wherein the at least one polyol is mannitol.

6. The method for manufacturing a single layer chewable tablet of claim 1, further comprising starch.

7. The method for manufacturing a single layer chewable tablet of claim 6, wherein the at least one polyol and starch are preformed into a mixture.

8. The method for manufacturing a single layer chewable tablet of claim 7, wherein the at least one polyol is about 70%-90% by weight and the starch is about 10%-30% by weight, relative to the total weight of the mixture.

9. The method for manufacturing a single layer chewable tablet of claim 1, further comprising a cyclodextrin, preferably wherein the cyclodextrin is selected from a group consisting of α-cyclodextrin, β-cyclodextrin, and γ-cyclodextrin, and, preferably wherein the cyclodextrin is about 5%-30%, 10%-25%, or 12%-20% by weight, relative to the total weight of the chewable tablet.

10. (canceled)

11. The method for manufacturing a single layer chewable tablet of claim 9, wherein the cyclodextrin is β-cyclodextrin.

12. (canceled)

13. The method for manufacturing a single layer chewable tablet of claim 1, wherein the chewable tablet remains stable up to 6 months under accelerated conditions of 40° C. and 75% relative humidity (RH), up to 12 months under conditions of 30° C. and 65% relative humidity (RH), and/or up to 12 months under conditions of 25° C. and 60% relative humidity (RH).

14. The method for manufacturing a single layer chewable tablet of claim 1, wherein the chewable tablet remains stable up to 24 months under conditions of 30° C. and 65% relative humidity (RH).

15. The method for manufacturing a single layer chewable tablet of claim 1, wherein the chewable tablet remains stable up to 36 months under conditions of 25° C. and 60% relative humidity (RH).

16. The method for manufacturing a single layer chewable tablet of claim 1, wherein the chewable tablet has a hardness of about 2-12, 3-11, or 4-10 kp.

17. The method for manufacturing a single layer chewable tablet of claim 1, wherein cetirizine is about 0.5%-20%, 0.5%-15%, 1%-10%, 1%-8%, 1%-6%, 1%-5%, by weight, relative to the total weight of the chewable tablet.

18. The method for manufacturing a single layer chewable tablet of claim 9, wherein the weight ratio of cetirizine and the cyclodextrin in the chewable tablet is about 1:1 to 1:10, 1:2 to 1:9, or 1:2.5 to 1:8.5.

19. The method for manufacturing a single layer chewable tablet of claim 1, further comprising additional pharmaceutically acceptable excipients.

20. The method for manufacturing a single layer chewable tablet of claim 19, wherein the additional pharmaceutically acceptable excipients comprise fillers, adsorbents, binders, disintegrants, lubricants, glidants, sweeteners, superdisintegrants, flavor and aroma agents, antioxidants, texture enhancers, or mixtures thereof.

21. The method for manufacturing a single layer chewable tablet of claim 20, wherein the fillers comprise monosaccharides, disaccharides, or mixtures thereof.

22. The method for manufacturing a single layer chewable tablet of claim 21, wherein the disaccharides are lactose monohydrates.

23-58. (canceled)
